(19) United States
(12) Patent Application Publication
(10) Pub. No.: US 2003/0083821 A1
(43) Pub. Date: May 1, 2003

(54) Fast secondary structure discovery method for protein folding

(76) Inventor: Eric C. Hannah, Pebble Beach, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 09/966,024

(22) Filed: Sep. 28, 2001

(51) Int. Cl. G06F 19/00; G01N 33/48; G01N 33/50

(52) U.S. Cl. 702/19

(57) ABSTRACT

A system and related methods are described for determining a three-dimensional protein structure. In certain embodiments, the methods include predicting the secondary structure for protein of known amino acid sequence, superimposing the secondary structure on a topomer model, and refining the topomer model. In other embodiments, a machine readable medium may provide instructions, which when executed by a machine cause said machine to perform a method including predicting a secondary structure for a protein of known amino acid sequence, superimposing the secondary structure on a topomer model, and refining the topomer model.

### FIELD OF THE INVENTION

The methods and systems described herein relate to the field of protein chemistry. In particular, they relate to the determination of three dimensional protein structures from amino acid sequences.

### BACKGROUND

A protein is a biopolymer in which anywhere from about fifty to several thousand amino acids may be connected together by peptide bonds, typically in a linear sequence that is referred to as the primary structure of a protein. Under physiological conditions, each protein spontaneously folds into a unique three-dimensional structure, known as the tertiary structure of a protein. Shorter domains of regularly folded sequences (alpha helices, beta sheets, and reverse turns) form the secondary structure of a protein. The native conformation of a protein, the tertiary structure, is closely related to its biological function. Hence, the prediction of protein conformation is not only of theoretical interest but is also of great importance for the design of novel drugs targeted against specific proteins and of synthetic proteins of specified function.

X-ray crystallography and Nuclear Magnetic Resonance (NMR) are two methods that are currently used to determine the tertiary structure of a protein. X-ray crystallography relies on the crystallization and X-ray diffraction of a subject protein. Only proteins isolated in or refolded into their native state in sufficient quantity and purity for crystallization can be used to determine the tertiary structure of a protein using X-ray crystallography. Additionally, the crystals of purified protein need to be stable and ordered to diffract X-rays. This method is difficult, time consuming and very expensive and has been of limited success for proteins with large hydrophobic domains, such as integral membrane proteins that may function as cell surface receptors or transporters. Proteins with large hydrophobic domains tend to form amorphous, non-crystalline precipitates due to the prevalence of hydrophobic interactions.

NMR is a solution-based approach for protein structure determination, based on the interactions between closely adjacent atomic nuclei. It is typically limited to analyzing the structures of smaller proteins, since larger proteins generate more complex and degenerate NMR spectra that are very difficult to resolve into protein structures. The inherent difficulties in these methods limit the speed at which three-dimensional protein structures can be obtained, as well as being biased against hydrophobic proteins. Only a few thousand well-characterized three-dimensional protein structures have been identified to date, with a small fraction of these being membrane-bound proteins.

Another method of three-dimensional protein structure determination is computational modeling. One method of modeling protein structure is to superimpose a target protein sequence onto the known three-dimensional structures of conserved regions in a family of related proteins. This approach is based on the general observation that proteins of similar amino acid sequence (primary structure) will often have similar secondary and tertiary structures. However, among the members of a given family there is often considerable variation in the conformations of regions located outside of structurally conserved regions. These variable regions contribute to the unique structural conformations of different proteins within a given protein family. The modeling of variable regions within protein families has been generally unsatisfactory. In many cases, target proteins may not fit within a well-defined family of closely related proteins, or there may be insufficient structural information on other members of the family on which to base a predicted structure. Also, the greater the difference in primary structure between two proteins, the less likely it is that a modeling approach based on sequence similarity between a target protein and a protein of known structure will be accurate.

Other methods of computational modeling have been attempted. In general, these have been of limited success for the prediction of protein conformation, particularly for the great majority of biologically important proteins that are greater than a few hundred amino acids in length. The rate at which amino acid sequences are being determined, especially amino acid sequences derived from the human genome project, greatly exceeds the rate at which three-dimensional protein structures can be determined by present methods. Advanced computational methods of determining a three-dimensional protein structure will speed the discovery of protein structure and function.

### BRIEF DESCRIPTION OF THE DRAWINGS

The following drawings form part of the specification and are included to further demonstrate certain embodiments. The disclosed methods and systems may be better understood by reference to one or more of these drawings in combination with the detailed description of specific embodiments presented herein.

FIG. 1A diagram representing an exemplary method.

FIG. 2A diagram representing an exemplary smart move method.

FIG. 3A diagram representing an exemplary method of secondary structure prediction.

FIG. 4A diagram representing an exemplary method of simulated annealing.

### DESCRIPTION OF ILLUSTRATIVE EMBODIMENTS

The biological properties of proteins, such as enzymatic activity, binding affinity for other proteins, and structural properties, depend directly on their three-dimensional structures. A method capable of determining the three-dimensional structure of a protein from its amino acid sequence would have numerous medical and industrial applications. For instance, such methods may be employed in the design of drugs that inhibit the activity of a protein or disrupt protein-protein interactions.

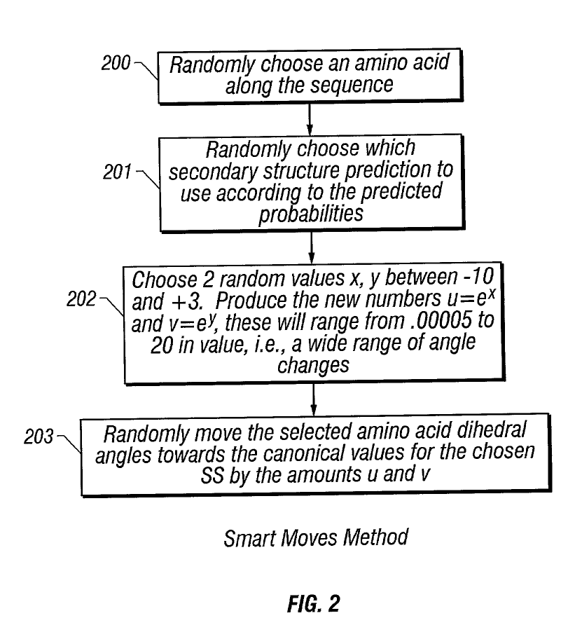

In certain embodiments, a unique combination of methods is used to determine the three-dimensional structure of a protein. The combination comprising a prediction of secondary structure for a target amino acid sequence and full atom protein modeling using topomers. As discussed below, short domains of secondary structure may be predicted with some accuracy based on the inherent tendencies of particular amino acid residues, or combinations of residues, to form alpha helices, beta sheets, or reverse turns. In certain embodiments, secondary structure prediction may be followed by energy minimization to refine a predicted secondary structure. The backbone dihedral angles may be adjusted to approximate the dihedral angle associated with the predicted secondary structure by using smart moves, as exemplified in FIG. 2. The use of smart moves may augment the speed and accuracy of the refinement of predicted secondary structure. The methods described may use at least one secondary structure prediction from one or more secondary structure prediction algorithms. Preferably a consensus secondary structure from a plurality of secondary structure prediction algorithms may be used.

In certain embodiments, refined secondary structure may be superimposed on a model protein structure, followed by refinement of a model to determine a three-dimensional protein structure. In certain embodiments, a “General Protein” topomer model, derived by topomer-sampling methods (Debe et al., Proc. Nat. Acad. Sci. USA, 96:2596-2601, 1999), can be used as a model protein structure. A predicted secondary structure(s) may be used to analyze and select an appropriate set(s) of topomers to use as a model protein structure.

Refinement of predicted secondary structure superimposed (modeled) onto a set(s) of topomers may be used to determine a three-dimensional protein structure. In certain embodiments simulated annealing may be used to refine a model protein structure. In particular embodiments, free energy minimization methods may be used to refine a model protein structure.

The three-dimensional structure of a protein may be represented by a protein structure file detailing the Cartesian coordinates of a majority of the atoms in the protein, preferably all atoms of the protein. The protein structure file may be accessed by computer systems running software that enables the viewing and modeling of a three-dimensional representation of a protein.

The methods described herein may be implemented by programming and executing the methods on a computer system. Certain embodied methods have as components secondary structure prediction, refinement of a predicted secondary structure, formation of a protein model based on secondary structure and/or topomers, and refinement of the model to determine the three-dimensional protein structure.

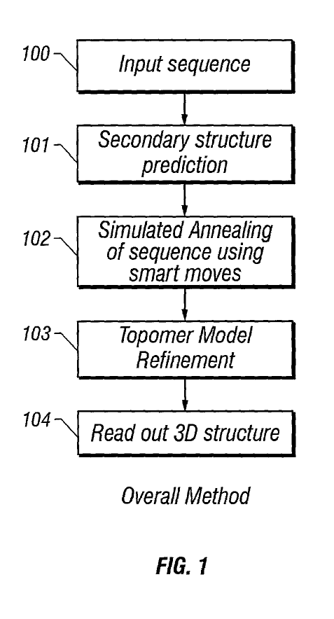

FIG. 1 is a flow chart illustrating an exemplary embodiment of the described methods. Block 100 represents the input of an amino acid sequence in an appropriate format, for example, single letter amino acid code in FASTA format. The amino acid sequence is then subjected to one or more secondary structure prediction methods represented by block 101, as discussed in detail below. The submission and retrieval of data from secondary structure prediction programs may be done manually or may be automated by a computer system. Block 102 represents the simulated annealing of the secondary structure prediction using smart moves and global energy minimization techniques. Full atom representations of the amino acid sequence in an aqueous environment can be generated by and stored on a computer system. A file representing the three-dimensional Cartesian coordinates of the predicted secondary structures can be written on computer readable media, for example a PDB file. The three-dimensional coordinates may then be submitted to programs accessible through a web browser by an Internet connection for further optimization by global energy minimization (see below). A secondary structure prediction submitted for energy minimization may be submitted with all secondary structural elements in linear order or each secondary structural element submitted individually and re-assembled after minimization.

Once a secondary structure has been optimized a three-dimensional protein structure may be determined by refinement of a topomer model of the protein incorporating the secondary structure as represented in block 103. The order of secondary structural elements may be used to choose subsets of general protein topomers for protein modeling. A general protein topomer comprises a reduced atomic and sequence independent representation of a specified length of amino acids with defined sets of dihedral angles for each amino acid. An assembly of predicted secondary structural elements will be consistent with a set(s) of general protein topomers. Based on secondary structure predictions general protein topomers consistent with the order of secondary structural elements may be defined. The topomer set may be refined as represented in block 103. Refinement of a topomer model may be carried out by using methods for three-dimensional structure refinement known in the art, examples of which are provided herein. Block 104 represents the readout of the three-dimensional protein structure after refinement of a topomer model. The atomic positions of the predicted structure can be stored as a file on computer readable medium. In one embodiment the readout is in Protein Database (PDB) format.

FIG. 2 is a diagram representing an exemplary smart moves method, corresponding to block 102 of FIG. 1. In certain embodiments, the diagram of FIG. 2 occurs after the protein's secondary structure has been predicted using one or more methods for secondary structure prediction. Block 200 represents randomly choosing an amino acid of the protein sequence. Block 201 represents choosing a secondary structure for the selected amino acid by secondary structure prediction. In certain embodiments, the secondary structure chosen may represent a consensus structure for the selected amino acid obtained from a comparison of multiple methods of secondary structure prediction. In alternative embodiments, as represented in block 201, a secondary structure for the selected amino acid may be chosen at random from any of the methods of secondary structure prediction. The selected secondary structure defines canonical values for the dihedral bond angles (phi and psi) flanking the peptide bond for a selected amino acid.

Starting with dihedral bond angles for an unfolded protein, values for dihedral bond angles may be varied for a selected amino acid. Block 202 represents randomly choosing values of X and Y between −10 and 3. X and Y are natural log exponents for the variables U and V, which represent changes in value of the dihedral bond angles for a selected amino acid. Using the limitations on X and Y, the generated values of U and V will range from 0.00005 to 20. This avoids biasing the simulated annealing towards small changes in dihedral bond angles by equalizing the probability that a large or a small change in dihedral bond angle will be utilized. Using randomly chosen X and Y, values of U and V are generated by the equations U=e^{x} and V=e^{y}. Block 203 represents the random movement of the dihedral bond angles for a selected amino acid towards the standard (canonical) values for the chosen secondary structure (ss) by the amounts U and V. This process is repeated throughout the length of the protein sequence until a sequence is annealed by achieving global energy minimization. Energy minimization values for the different conformations defined by each set of dihedral bond angles may be determined by standard techniques and methods as discussed below.

In certain embodiments of the method illustrated in FIG. 2, the secondary structural elements may include all possible forms of secondary structure. In alternative embodiments, probability values may be generated for alpha helical formation. The latter approach focuses on identification of alpha helical domains, which are known to form very early in protein folding. In either case, energy minimization and smart moves are used to refine the predicted secondary structure of the target protein.

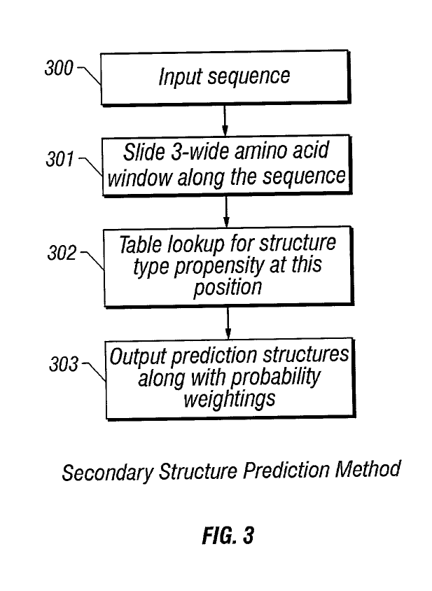

FIG. 3 is a diagram illustrating an exemplary method for secondary structure prediction. Block 300 represents the input of an amino acid sequence in appropriate format, such as single letter amino acid code FASTA format. Block 301 represents an embodiment that uses a three amino acid window to predict the propensity of an amino acid to form a particular secondary structure. The window calculates a moving average for the probability of formation of each type of secondary structure at a given amino acid position, based on the type of amino acid and its two nearest neighbors. A program to perform such a method, for example the Chou-Fasman method, may be accessed by a web browser through the Internet. The propensity of an amino acid to form a particular structure will be listed in a table as represented by block 302. Such programs typically output a predicted secondary structure with associated probability weightings for each amino acid as represented in block 303. An output will generally be sent from the server associated with the program by email to an email account designated at the time of submission. A typical output comprises a listing of a linear amino acid sequence and associated predicted secondary structure(s), along with probability values. Dihedral backbone angles can be adjusted to the appropriate angles associated with the predicted secondary structure(s).

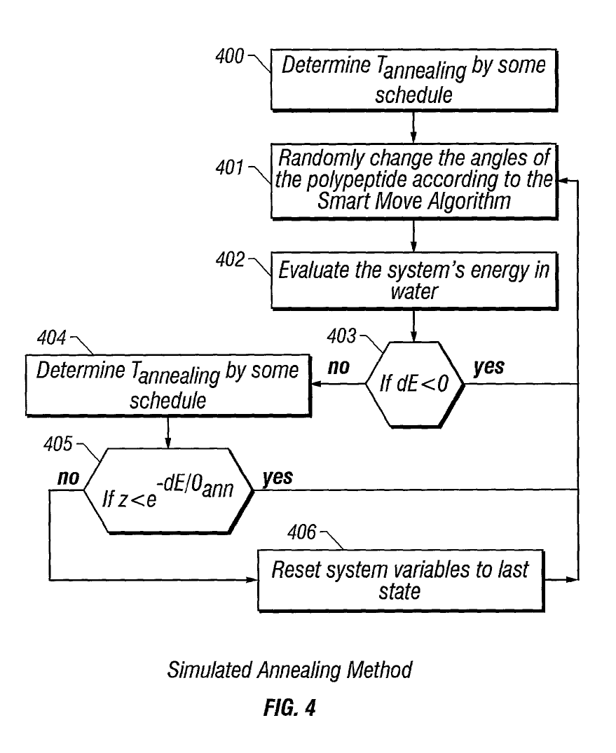

FIG. 4 is a diagram illustrating an exemplary method of simulated annealing. In this method, a protein is assumed to be heated above a temperature at which it melts into a random coil type of configuration. The temperature is slowly lowered until the protein refolds back into its native conformation (anneals). Depending on a predicted protein conformation, the value of the calculated annealing temperature will increase or decrease, representing an increase or decrease in the energy state of the protein (i.e., energy minimization). Block 400 represents the determination of the temperature (T) of annealing according to a standard schedule as known in the art (see below). Block 401 represents the random changing of dihedral bond angles using a smart move method, as illustrated in FIG. 2. Block 402 represents the evaluation of the total energy of the system in an aqueous environment, using the new values for dihedral bond angles. Block 403 represents a decision point from which the change in energy of a model protein conformation is analyzed. If the total calculated energy is less than the starting total energy then go to block 401 and repeat the method. If the total calculated energy is greater than the starting total energy then continue to block 404. Block 404 represents a decision point that may be used to traverse local energy minima and allows the method to progress to an overall minimum energy. The random acceptance of a total calculated energy that is greater than the starting total energy may allow the method to escape a local energy well and continue progress to a lower energy state. If a random number between 0 and 1 is less than this quantity, the new conformation is accepted. If the new conformation is accepted then go to block 401 and repeat the method. If the new confirmation is rejected using this calculation then go to block 406, which represents the resetting of the system to the last state and begin from block 401.

Secondary Structure

Secondary structures are regular structural elements that are formed by hydrogen bonding between various segments of a protein. Although hydrogen bond interactions may occur between amino acid residues that are spatially close together, they may also involve residues that are far apart along the peptide backbone. Different types of secondary structures may involve a small portion of the total protein structure, or they may comprise a majority of the residues in the protein, for example in beta-barrel type proteins.

Generally, there are three types of secondary structural elements: helices, beta-sheets and reverse turns. In addition, regions of a protein that do not adopt a regular secondary structure may form random coils. Alpha helices are a type of helix formed by hydrogen bonding between a backbone carbonyl group of one amino acid and a backbone amino group of the fourth amino acid residue along the peptide backbone of a protein. This is a common type of helical structure and it is compatible with all amino acids except proline. Other types of helices are known, such as the 3₁₀ helix or the π helix. The amino acid side-chains are generally positioned on the outer surface of helices. A beta-sheet is formed by hydrogen bonding between two or more beta strands, in which the peptide backbone adopts an extended conformation with the side chains pointing up or down relative to the plane of the beta-sheet. Beta-sheets may be parallel, anti-parallel, or mixed beta-sheets. Parallel beta sheets contain beta-strands running in the same amino terminal to carboxy terminal directions. Anti-parallel beta-sheets contain beta-strands running in opposite directions. A mixed beta-sheet contains both parallel and anti-parallel beta-strands. Unlike alpha helices, the hydrogen bonding residues in beta-strands need not be close together along a peptide backbone in order to form hydrogen bonds. Reverse turns are short secondary structures that enable the protein backbone to turn 180 degrees and may be stabilized by hydrogen bonding between the n and n+3 amino acids of the turn. Random coils are segments of a protein that are not characterized by regular hydrogen bonding patterns. Coils may take the form of unstructured loops or terminal portions of the amino acid chain.

Secondary structure prediction is based on the propensity of individual amino acids to form a particular secondary structure. Any given amino acid will have a statistical probability of forming a helix, beta-sheet, reverse turn or random coil. Probabilities may be empirically determined by analyzing known protein structures and small peptides in solution and determining, for any given amino acid, how often it is found in an alpha helix, a beta-sheet, a reverse turn or a random coil. This information has been obtained for each of the 20 naturally occurring amino acids. Thus, secondary structure predictions may be used to estimate probabilities of finding alpha helices, beta strands, reverse turns, or random coils at each amino acid within the sequence of a protein or a family of related proteins. Associated with the predicted secondary structure for each amino acid residue within a protein sequence are a set of favorable dihedral angles (one on each side of the peptide bond).

Some secondary structure prediction methods are single sequence methods, which rely on a single polypeptide sequence for prediction of secondary structure. Other secondary structure prediction methods are multi-sequence methods and use alignments of a plurality of related protein sequences. Single sequence methods include those of Chou and Fasman; and Gamier, Osguthorbe and Robson (GOR). The Chou-Fasman method uses a sliding window of three adjacent amino acids to calculate secondary structure probabilities and gives a fairly reliable indicator of the general locations of alpha helices. The GOR method uses the sum of propensities of all residues within a seventeen amino acid window to more accurately account for the effect of neighboring residues along the peptide backbone. These programs are based on statistical probabilities and simple rules developed from observation of known protein structures.

Multi-sequence methods use aligned sequences for prediction of secondary structure. The patterns of amino acid substitutions in homologous proteins are used to derive information on probable secondary structures. An exemplary multi-sequence method is PROFsec, available on the PredictProtein server. PROFsec is based on a two-layer feed-forward neural network. Aligned sequences with known secondary structure are used to train the neural network, which can then be used to predict the secondary structure of aligned protein sequences of unknown secondary structure. In one layer the occurrence of various residues in a thirteen amino acid window is correlated with the secondary structure of the central residue. In a second layer the output from the first layer in a seventeen amino acid window is used to predict the secondary structure at a central amino acid.

Many of the secondary structure prediction programs are accessible by a web browser through the Internet. These programs include, but are not limited to Chou-Fasman, GOR, PSI-pred, JPRED, Prof, PREDATOR, PHD, ZPRED, nnPredict, BMERC PSA Server, SSP, and the like. Generally, these programs accept the amino acid sequences in one-letter amino acid code that is in FASTA format, PIR, plain text, or similar formats. For example, a sequence in FASTA format begins with a single-line description, followed by lines of sequence data. The description line is distinguished from the sequence data by a greater-than (“>”) symbol in the first column. It is recommended that all lines of text be shorter than 80 characters in length. An example sequence in FASTA format is:

>Sequence description
ELRLRYCAPAGFALLKCNDADYDGFKTNCSNVSVVHCTNLMNTTVTTGLL
LNGS

Another example of a commonly used format is the PIR format. A sequence in PIR format consists of one line starting with a “>” sign, followed by a two-letter code describing the sequence type (P1 (protein sequence), F1, DL, DC, RL, RC, or XX), followed by a semicolon, followed by the sequence identification code (the database ID-code). One line contains a textual description of the sequence. One or more lines contain the sequence itself. The end of the sequence is marked by an “*” (asterisk) character. A file in PIR format may comprise more than one sequence. The PIR format is also often referred to as the NBRF format.

Sequences are expected to be represented in the standard IUB/IUPAC amino acid codes, with these exceptions: lower-case letters are accepted and are mapped into upper-case; a single hyphen or dash can be used to represent a gap of indeterminate length; and in amino acid sequences, U and * are acceptable letters. Any numerical digits in the sequence should either be removed or replaced by appropriate letter codes (e.g., X for unknown amino acid residue). The accepted amino acid codes are: A alanine, P proline, B aspartate or asparagine, N asparagine, Q glutamine, C cysteine, R arginine, D aspartate, S serine, E glutamate, T threonine, F phenylalanine, U selenocysteine, G glycine, V valine, H histidine, W tryptophan, I isoleucine, Y tyrosine, K lysine, Z glutamate or glutamine, L leucine, X any, M methionine, * translation stop, N asparagine, and “−” gap of indeterminate length. Amino acid sequences are generally submitted for secondary structure analysis by email or a web browser.

In certain embodiments one or more secondary structure prediction programs may be used to derive a consensus secondary structure for a target amino acid sequence. A preferred embodiment is to analyze the primary sequence of the protein with a plurality of secondary structure prediction methods to derive a consensus prediction. A consensus prediction may be derived by assigning a feature to a particular region of the amino acid sequence if the feature is present in a majority of secondary structure predictions. In certain embodiments, a consensus secondary structure is obtained from the JPRED server. Other embodiments may refine a secondary structure prediction multiple times to generate an ensemble of refined secondary structures for protein modeling. In further embodiments, regions of good beta sheet potential may also be included.

Bond Angles and Models of the Amino Acid Sequence

A protein has a sequence of repeating backbone groups that form a polypeptide backbone, consisting generally of primary amine (N—H), alpha carbon (Cα), and carbonyl (C═O) groups. Each pair of adjacent amino acids along a polypeptide chain is connected by a peptide bond. Peptide bonds are formed by a condensation reaction between an amine group of a first amino acid and a carboxyl group of a second amino acid. The peptide bond has an approximately planar geometry, a bond angle of approximately 180 degrees, due to its partial double bonded character. The backbone of a protein is a sequence of planar peptide bonds linked by Cα atoms. Changes in backbone conformation result from rotation about the non-peptide bonds, which are a nitrogen-alpha carbon bond (N—Cα) and an alpha carbon-carbonyl carbon bond (Cα—C). A dihedral angle is the angle formed by these bonds, with the N—Cα bond defined as the phi (φ) angle and the Cα—C bond defined as the psi (ψ) angle. The peptide backbone conformation, and in turn the tertiary structure of the protein, is defined by the sum of the dihedral angles about each alpha carbon. In certain embodiments, refinement of protein structure occurs by varying the values of phi and psi for each amino acid in the protein and determining the effect of the change in bond angle on either the total energy of the protein conformation in aqueous solution or the entropy of the system.

Sterically allowable values of the dihedral angles for each type of amino acid can be represented by a Ramachandran diagram. A Ramachandran diagram may be determined by calculating the distance between the atoms of a tripeptide at all values of phi and psi. A number of dihedral angle conformations will be prohibited due to resulting interatomic distances that are less than the corresponding van der Waals distances of the respective atoms. If the interatomic distance is less than the van der Waal distance then repulsive forces between atoms becomes too great and the phi/psi conformation is not possible. Thus, a number of conformations resulting from specific combinations of phi and psi bond angles will be sterically prohibited. Typically, the values of phi and psi for protein secondary structures fall within allowed values of a Ramachandran diagram. For each amino acid in a protein chain, there will be multiple combinations of phi and psi that are accessible and which are consistent with one or more types of secondary structure.

In certain embodiments all permissible values for all phi and psi angles in the protein backbone may be used during the refinement of the protein structure. In other embodiments the values of phi and psi used for each amino acid residue may be limited to the dihedral angles consistent with the predicted secondary structure of that residue. In particular embodiments phi and psi angles may be randomly chosen according to a log distribution of angle choices so that small changes are as probable as very large changes in dihedral angle. Typically, phi and psi angles that are randomly chosen according to a log distribution will accelerate convergence to a final secondary structure, such as a helix.

Optimization of Secondary Structure

In certain embodiments, predicted secondary structure will be optimized by minimizing the energy of secondary structural elements. A variety of methods are known for minimizing the energy of a molecular structure. These include, but are not limited to random Monte Carlo methods with or without simulated annealing, genetic algorithms, Brownian dynamics, and other similar methods. Such methods may be used for the refinement of a predicted protein structure as well. A graphical representation of the potential energy surface for a predicted secondary or protein structure may be characterized by a set of local minima, saddle points and a global energy minimum. The goal of an energy minimization process is to arrive at a global energy minimum and avoid becoming trapped in a local energy minimum. During the optimization of predicted secondary or protein structure for a target protein, a number of possible conformations are explored and a number of local minima will be traversed to arrive at the global minimum.

Simulated Annealing

Simulated annealing is a general optimization method that simulates the slow cooling of a physical system, such as an unfolded protein in aqueous solution. There is a cost function associated with the state of the system, which can be changed in various ways. The energy of a protein conformation may be calculated by using a molecular force field, as described below, at each temperature evaluated. Simulated annealing works by iteratively proposing changes and either rejecting or accepting the change based on the change in the cost function, these changes may be carried out using random Monte Carlo methods, as described below. For protein folding, the change might be in the values of phi and psi for a selected amino acid. Typically the acceptance or rejection of the change is governed by the change in energy of the system. If the energy of the system decreases then the change is accepted and the next change may be selected and analyzed. However, a strict rule that accepted only decreases in energy and prohibited any changes resulting in an increase in energy could result in the folding solution becoming trapped in a local energy minimum. This is because once the conformation enters the local energy minimum well, it may not exit the local minimum. To prevent this occurrence, many methods of simulated annealing allow for occasional changes that result in an increase in the total energy of the system (see FIG. 4, blocks 403-405). A change that results in an increase in total energy may be accepted if it meets the conditions of a Boltzman probability function (FIG. 4, block 405) Otherwise the change is rejected and the initial conformation is used to select a new set of values for phi and psi.

In the context of protein folding, simulated annealing refers to a hypothetical process in which the protein is first melted and then allowed to cool by slowly reducing the temperature. The atoms of the protein attempt to arrange themselves into a lower energy state as the system is cooled. Collective energy states of the all atoms in a protein can be considered as a function of the conformation of the protein. The probability that an atom will be at any energy level can be calculated by use of the Boltzmann distribution. As the temperature of the protein decreases, the Boltzmann distribution tends toward the atomic configuration that has the lowest energy. The thermal equilibrium process may be simulated at a fixed temperature by Monte Carlo methods to generate a series of energy states. In such a method, the system is perturbed (by manipulation of dihedral bond angles) to yield a new configuration of the atoms. The energy level before perturbation (Es) and the energy level after perturbation (Ei) are compared. If Es is greater than Ei (i.e., δE<0), the changed system is accepted as the new configuration of the protein. If δE>0, the probability of accepting the change is as described in FIG. 4, block 404 and 405. One may loop through the atoms sequentially or in random order. In particular embodiments phi and psi angles can be randomly chosen according to a log distribution of angle choices so that small changes are as probable as very large changes in angle. In certain embodiments a smart move may be used, particularly a smart move as outlined in FIG. 2.

Molecular Mechanics

Molecular mechanics is a computational method designed to give accurate structures and energies of molecules. It treats molecules as collections of masses that are interacting with each other by harmonic or other forces between bonded atoms and by van der Waals and electrostatic forces between non-bonded atoms. Mathematical functions of the atomic coordinates called potential energy functions may be used to describe these interactions. Various parameters derived from experimental observations may be included in the potential energy function, also known as a force field.

In general, a force field method is the calculation of molecular conformational geometries and energies using a combination of empirical force fields or potential functions (Burkert, U., & Allinger, N. L. (1982). Molecular Mechanics, ACS Monograph 177, American Chemical Society, Washington, D.C.). An assumption is typically made on bond lengths and angles, deviations from which result in bond and angle strain respectively. Repulsive or attractive van der Waals and electrostatic forces between nonbonded atoms can be taken into account, as well as other molecular forces. The basic idea of molecular mechanics is that simple molecules have “natural” bond lengths and bond angles. Any structural deviation from such “ideal” molecular geometry will result in an increase in potential energy. One of the fundamental assumptions of molecular mechanics is that the total potential energy of a molecule can be divided into several parts. A typical potential energy function form widely used for proteins is:

$$E ( R ) = \frac{1}{2} {Σ}_{b o n d s} {{k}_{b} ( b - {b}_{o} )}^{2} + \frac{1}{2} {Σ}_{a n g l e s} {{k}_{θ} ( θ - {θ}_{o} )}^{2} + \frac{1}{2} {Σ}_{t o r s i o n a l} {k}_{ω} [ 1 + cos ( n ω - δ ) ] + Σ ( A / {r}^{12} - B / {r}^{6} + {q}_{1} {q}_{2} / ∈ ) \qquad equation ( 1 )$$

where E(R) is a function of the coordinate set, R, of all the atoms in the system. The first term corresponds to a Hooke's law description of bond stretching. The second term is a similar approximation to the energy of bond angle bending. The parameters k_{b}and k_{q} are force constants that determine the flexibility of the bonds, b₀ and q₀ are natural bond length and bond angle, while b and q are the actual bond length and bond angle. The third term accounts for the energy associated with torsional angle rotations. The last term represents the non-bonded interactions between two atoms separated by distance r. It has three parts: the first two are the Lennard-Jones 6-12 potential which includes both short-distance repulsive and long-distance attractive interactions, and the last one corresponds to the electrostatic energy where q₁ and q₂ are the charges on atoms 1 and 2. Parameters A and B depend on the atoms involved and e is the dielectric constant of the medium.

Force fields are mathematical representations of a potential energy function used in the computation of the energy of a protein structure. For instance, in simulated annealing a force field may be used to calculate the energy of protein conformation after a random move in a Monte Carlo method at a particular temperature. Understanding, analyzing, and predicting three-dimensional structural models of proteins including their conformations, binding affinities, and related properties, are typically provided for by the application of various force fields.

In an exemplary embodiment the molecular force field AMBER (Assisted Model Building with Energy Refinement; may be used to predict the energy of the polypeptide. AMBER is the collective name for a suite of programs that allow users to carry out molecular dynamics simulations, particularly on biomolecules. Alternative programs and/or program suites that may be used for the computation of molecular force fields and simulated annealing include, but are not limited to, X-PLOR (Yale University, New Haven, Conn.), INSIGHTII (Molecular Simulations Inc., San Diego, Calif.), CHARMM (Harvard University, Cambridge, Mass.), DISCOVER (Molecular Simulations Inc., San Diego, Calif.), GROMOS (ETH Zurich, Zurich, Switzerland), and similar programs.

In another exemplary embodiment CHARMM (Chemistry at Harvard Macromolecular Mechanics) may be used. CHARMM is a program for macromolecular dynamics and mechanics. It performs standard molecular dynamics in many different ensembles using algorithms for timestepping, long-range force calculation and periodic images. CHARMM may be used for energy minimization, normal modes and crystal optimizations as well. The potential energy functions available for use with CHARMM have been extensively parameterized for simulations of proteins, nucleic acids and lipids. Free energy methods for chemical and conformational free energy calculations are also fully developed and available in CHARMM.

Once a potential function is chosen, another factor to consider in a molecular mechanics simulation is how the minimum energy conformation is determined. The landscape of a potential energy surface as a function of the coordinates of all the atoms in a system has many peaks (local maxima) and valleys (local minima). Each valley corresponds to a stable or semistable state of the system. For a protein, the structure associated with a stable state is called a conformation. Therefore, conformations can be found by locating the local minima on a potential energy surface. The computational method that starts with a set of atomic coordinates of the system and finds a nearby potential energy local minimum is call energy minimization. Various energy minimization methods are available. The methods using the first-derivatives of the potential energy function are usually less computationally intensive, while higher accuracy can often be achieved by using the methods involving both the first- and second-derivatives of a potential energy function.

The fundamental idea of predicting the structure of a protein using molecular modeling relies on the assumption that the conformation with the lowest potential energy is the native conformation of the protein. Therefore, the task of finding the native structure of a protein becomes the search for the global potential energy minimum.

Molecular Dynamic and Monte Carlo Simulations

Molecular dynamics is a computational method for simulating the motion of a system of many particles. It requires knowledge of the interaction potential from which the forces acting on each particles can be calculated, and the equations of motion that govern the dynamics of the particles. Molecular mechanics force fields are often used as the potential functions in molecular dynamics simulations. The force on atom i is calculated from the derivatives of the potential energy function with respect to the position of atom i (dE/dxi, dE/dyᵢ, dE/dzᵢ). Newton's equation, fᵢ=mᵢaᵢ, is used for finding the accelerations of each particles at each simulation step. More details of the methodology of molecular dynamics and its applications in biology may be found in van Gunsteren, W. F., & Berendsen, H. J. C. (1990). Computer simulation of molecular dynamics: Methodology, applications, and perspectives in chemistry. Angew. Chem. Int. Ed. Engl. 29, 992-1023.

The total energy of a system is the sum of both potential energy and kinetic energy. The mean kinetic energy is related to the temperature T of the system by

$$\frac{1}{2} {Σ}_{n t o i = 1} {m}_{i} ( {v}_{i}^{2} ) = 3 / 2 N {k}_{B} T \qquad ( 2 )$$

where N is the total number of atoms in the system, vᵢ² is the average velocity squared of the ith atom and k_{B} is the Boltzmann constant.

Equation 2 may be used to control the temperature of the system. Simulated annealing, as described above, is a technique where the simulated protein system starts at a high temperature, and then is cooled down gradually. By heating the protein to a high temperature, the simulation enables it to overcome larger energy barriers and to sample more conformations of interest. Ideally, as the system is cooled towards 0° K., the protein is trapped in the global minimum energy conformation. If the force field used in the simulation has sufficient accuracy, this global minimum energy conformation should be close to the native structure of the protein. Metropolis et al. developed a Monte Carlo method, as described above, for randomly searching the conformational space that simulates a molecular system by randomly changing its conformation. The energy of each new random conformation may be compared to the energy of the previous one. If the new energy is lower, then the new structure becomes the current conformation. If the new energy is higher, then the value of the Boltzmann factor is compared to a random number between 0 and 1. If the Boltzmann factor is greater than the random number, then the new structure becomes the current conformation.

The advantage of a Monte Carlo method is that its randomness can overcome many energy barriers. On the other hand, for the same reason, simulations using Monte Carlo methods are usually slower to converge than those using molecular dynamics. Simulated annealing can be carried out in a Monte Carlo just as in a molecular dynamic simulation.

Topomers

A topomer is a group of general protein or protein conformations that share the same backbone topology (Debe et al., Proc. Nat. Acad. Sci. USA, 96:2596-2601, 1999). The topology of a protein defines the connections, relative positions, and organization of secondary structural elements within three-dimensional space. A set of all topomers for a protein with a specified number of amino acids may be generated. Once these topomers are generated the secondary structure derived from prediction methods can be modeled onto a subset of the topomers that contain similar secondary structural elements in similar order.

Briefly, a set of general protein topologies may be produced by computational determination of all possible folds of a polypeptide backbone for a protein of a specified length (i.e. the length of the target protein). The model general proteins are independent of amino acid sequence, thus resulting topomers will be the set of candidate structures for any protein having the same number of amino acid residues. The groups of topomers are typically derived by using Continuous Configuration Boltzman Biased Direct Monte Carlo Method as described by Sadanobu and Goddard (J. Chem. Phys. 106:6722, 1997). A secondary structure, as described above, may be mapped onto a set of topomers to form a model structure and a model structure refined to determine a three dimensional conformation.

A refined partially folded secondary structure, particularly optimized full atom secondary structures, may be superimposed on a selected subset of general protein topomers. In certain embodiments, an initial subset of topomers may be identified onto which the refined secondary structure may be modeled, similar to homology modeling. The pattern(s) of secondary structure typically will be used to reduce the number of topomers used for modeling. The embodied methods use a novel combination of techniques to enhance topomer modeling for minimization and refinement of a three-dimensional protein structure.

Protein Structure Determination

Refinement of a three-dimensional protein structure derived from a secondary structure prediction(s), which may or may not be refined, modeled onto a topomer will typically comprise optimization of the topomer model. In certain embodiments, protein structure determination comprises alignment of secondary structure with topomer backbones (secondary structure-topomer alignment), model building, and protein refinement. Refinement of a protein model to determine a three-dimensional protein structure may be performed by using simulated annealing of a model structure to determine the structure with the minimum free energy. A variety of programs, including but not limited to AMBER, CHARMM, X-PLOR, INSIGHTII, as well as other programs described above, and the like can be used for structure refinement.

Files containing the information for three-dimensional protein structures are typically in Protein DataBase format (PDB), Molecular Modeling Database format (MMDB), or similar file formats. The files typically comprise the Cartesian coordinates of each atom in the molecule. For example, PDB format list the characteristics of a protein structure using ACII and special characters. Every PDB file may be broken into a number of lines terminated by an end-of-line indicator. Each line in the PDB entry file consists of 80 columns. The last character in each PDB entry should be an end-of-line indicator. Each line in the PDB file is self-identifying. The first six columns of every line contain a record name, left-justified and blank-filled. This must be an exact match to one of the stated record names. The PDB file may also be viewed as a collection of record types. Each record type is detailed in the PDB format description.

The methods described can be used to analyze an amino acid sequence and determine a three-dimensional protein structure encoded by an amino acid sequence. The output of such an analysis will typically be a set of Cartesian coordinates representative of a majority of the atoms of a target protein.

Target Protein Sequences

Proteins are linear amino acid polymers, or polypeptides. Proteins can be composed of twenty different types of amino acids. The linear sequence of amino acid residues determines the primary structure of a protein. The primary structure of a protein can be elucidated using standard methods of direct protein sequencing, experimental gene determination, computational gene prediction, or a combination of these and other techniques.

Direct Sequencing of Isolated Proteins.

The primary sequence of proteins can be directly determined by a stepwise chemical degradation process in which single amino acids are removed one by one from the end of a protein and identified. Edman degradation is a standard method for protein sequencing. In Edman degradation amino acid removal from the end of the protein is accomplished by reacting the N-terminal amino acid residue with a reagent, which allows selective removal of that residue. The resulting amino acid derivative is converted into a stable compound, which can be chemically removed from the reaction mixture and identified. Very small amounts of protein can be sequenced by reverse phase high pressure liquid chromatography using a UV detector. Alternate methods of detecting released amino acids include radiolabeling of a peptide or reagent, radiolabeling during synthesis of the polypeptide, and other enhanced detection methods.

Computational Gene Prediction

Computational gene prediction typically involves the analysis of nucleic acids to determine the amino acid sequence of a protein. The sequence of both DNA and RNA can be analyzed to determine the primary structure of a protein. Usually messenger RNA is reverse transcribed to form a complementary DNA (cDNA). The cDNA contains a sequence of three letter codes (codons) that designate the sequence of amino acids in the protein. Genomic DNA sequences can be manipulated by computer algorithms to predict the presence of a gene in genomic DNA sequence or predict the proper reading frame of a cDNA sequence. These computational gene prediction methods are used extensively in the search for coding regions.

Ab initio prediction is far from perfect, and adding other evidence can improve computational gene prediction. Additional evidence that may strengthen the results of the computer methods for gene identification are database searching for corresponding expressed sequence tags (ESTs); experimental gene determination including RT-PCR amplification, exon trapping, northern blotting, and other experimentally based techniques.

Many programs are available for the detection and discovery of genes, coding regions, and open reading frames in genomic and cDNA sequences. Exemplary programs that may be used to identify these sequences for the determination of the encoded amino acid sequence include, but is not limited to NCBI ORF finder; BCM genefinder; BCM search launcher: gene feature searches; Splice prediction by neural network; Genie; GRAIL and other similar programs. Many of these systems are still under development, and improvements continue to appear. Some of the leading systems are GRAIL, GeneID, GeneParser, SORFIND and FGENEH. Gene finding programs, such as GRAIL, GeneID, GeneParser, GenLang, FGENEH, Genie, and EcoParse use neural nets and other artificial intelligence or statistical methods to locate genes in DNA sequences.

Experimental Gene Prediction

Experimental gene determination typically uses isolated nucleic acids to determine which portion of a gene is transcribed and translated into a protein. DNA and RNA can be isolated using conventional methods as described in Sambrook, Fritsch, Maniatis, Molecular Cloning: A Laboratory Manual, 2nd Ed., Cold Spring Harbor Press, Cold Spring Harbor, N.Y., 1989. Messenger RNA (mRNA), may be reverse transcribed and cloned as a complementary DNA or cDNA. Genomic DNA clones and subclones or portions thereof can be used as probes for RNA blotting to identify genomic regions that are present in messenger RNA or as a template in exon trapping experiments to capture potential exon or coding sequences by translation and splicing of a vector in a cell culture system. These and other conventional methods, such as cDNA selection and similar experimental procedures may be used to directly or indirectly determine the amino acid sequence of a target protein.

Information Processing and Control System and Data Analysis

In certain embodiments, methods of three-dimensional protein structure determination may be interfaced with an information processing and control system. In an exemplary embodiment, the system incorporates a computer comprising a bus or other communication means for communicating information, and a processor or other processing means coupled with the bus for processing information. In one embodiment, the processor is selected from the Pentium(r) family of processors, including the Pentium(r) II family, the Pentium(r) III family and the Pentium(r) 4 family of processors available from Intel Corp. (Santa Clara, Calif.). In alternative embodiments, the processor may be a Celeron(r), an Itanium(r), or a Pentium Xeon(r) processor (Intel Corp., Santa Clara, Calif.). In various other embodiments, the processor may be based on Intel(r) architecture, such as Intel(r) IA-32 or Intel(r) IA-64 architecture. Alternatively, other processors may be used.

The computer may further comprise a random access memory (RAM) or other dynamic storage device (main memory), coupled to the bus for storing information and instructions to be executed by the processor. Main memory may also be used for storing temporary variables or other intermediate information during execution of instructions by processor. The computer may also comprise a read only memory (ROM) and/or other static storage device coupled to the bus for storing static information and instructions for the processor. Other standard computer components, such as a display device, keyboard, mouse, modem, network card, or other components known in the art may be incorporated into the information processing and control system. The skilled artisan will appreciate that a differently equipped information processing and control system than the examples described herein may be desirable for certain implementations. Therefore, the configuration of the system may vary within the scope of the invention.

In particular embodiments, the detection unit may also be coupled to the bus. Data from the input of amino acid sequence may be processed by the processor and the processed and/or raw data stored in the main memory. Data on known secondary structures known for amino acid sequences may also be stored in main memory or in ROM, as well as output from web browser accessible programs. The processor may analyze the data from secondary structure prediction(s) and protein modeling to determine a three-dimensional protein structure. The information processing and control system may further provide automated control of the exchange of information between the processing unit and the prediction and modeling programs.

It should be noted that, while the processes described herein may be performed under the control of a programmed processor, in alternative embodiments, the processes may be fully or partially implemented by any programmable or hardcoded logic, such as Field Programmable Gate Arrays (FPGAs), TTL logic, or Application Specific Integrated Circuits (ASICs), for example. Additionally, the methods described may be performed by any combination of programmed general-purpose computer components and/or custom hardware components.

In certain embodiments, custom designed software packages may be used to analyze the data obtained from the structure prediction and modeling programs. In alternative embodiments, data analysis may be performed using an information processing and control system and publicly available software packages. Non-limiting examples of available software for secondary structure analysis includes Chou-Fasman, PROFsec, and a variety of software packages available through the JPRED server at. Non-limiting examples of available software for molecular modeling includes AMBER, CHARMM, X-PLOR, INSIGHTII, and a variety of software packages available through the National Institutes of Health website at.

All of the COMPOSITIONS, METHODS and APPARATUS disclosed and claimed herein can be made and executed without undue experimentation in light of the present disclosure. While the compositions and methods have been described in terms of preferred embodiments, it will be apparent to those of skill in the art that variations may be applied to the COMPOSITIONS, METHODS and APPARATUS and in the methods described herein without departing from the concept, spirit and scope. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the described embodiments as defined by the appended claims.

What is claimed is:

1. A method comprising:
predicting a secondary structure of a protein;
superimposing the predicted secondary structure on a set of topomers;
refining the superimposed secondary structure; and
predicting a tertiary structure of a protein

2. The method of claim 1, wherein said secondary structure is a consensus secondary structure prediction.

3. The method of claim 1, further comprising annealing the secondary structure by energy minimization.

4. The method of claim 3, wherein said energy minimization is by a random Monte Carlo method.

5. The method of claim 4, wherein the random Monte Carlo method uses random moves from a log probability table.

6. The method of claim 3, wherein the random Monte Carlo method uses smart moves.

7. The method of claim 1, wherein the secondary structure superimposed on a set of topomers is refined by energy minimization.

8. The method of claim 7, wherein the secondary structure superimposed on a set of topomers is refined using a molecular modeling program.

9. The method of claim 8, wherein the molecular modeling program is X-PLOR.

10. A method comprising:
predicting a secondary structure of a protein;
generating a three-dimensional representation of the predicted secondary structure;
optimizing the secondary structure by adjusting dihedral angles using smart moves; and
determining a three-dimensional protein structure by modeling the optimized secondary structure on a topomer model.

11. The method of claim 10, wherein said secondary structure is a consensus secondary structure prediction.

12. The method of claim 10, wherein optimization is performed using a random Monte Carlo method.

13. The method of claim 12, wherein the random Monte Carlo method is used in conjunction with a localized energy function.

14. The method of claim 10, wherein said three-dimensional structure model is refined using simulated annealing.

15. A machine readable medium that provides instructions, which when executed by a machine cause said machine to perform a method comprising:
predicting a secondary structure of protein;
superimposing the secondary structure on a topomer model; and
refining the topomer model.

16. A machine readable medium as in claim 15, wherein said secondary structure is a consensus secondary structure prediction.

17. A machine readable medium as in claim 15, further comprising energy minimization of the secondary structure prediction.

18. A machine readable medium as in claim 17, wherein said energy minimization is by a random Monte Carlo method.

19. A machine readable medium as in claim 18, wherein random moves are selected from a log probability table.

20. A machine readable medium as in claim 15, wherein the topomer model is refined by topological entropy minimization.

21. A machine readable medium as in claim 20, wherein the topomer model is refined by a molecular modeling program.
